(19) United States
(12) Patent Application Publication
(10) Pub. No.: US 2013/0123716 A1
(43) Pub. Date: May 16, 2013

(54) Vaginal Therapeutic Device Including Copper Metal

(71) Applicants: Chun Lim Abbott, Pittsburgh, PA (US); Dominic C. Abbott, Pittsburgh, PA (US)

(72) Inventors: Chun Lim Abbott, Pittsburgh, PA (US); Dominic C. Abbott, Pittsburgh, PA (US)

(21) Appl. No.: 13/464,005

(22) Filed: May 4, 2012

Related U.S. Application Data
(63) Continuation-in-part of application No. 13/317,230, filed on Oct. 12, 2011.
Application No. 13/317,230 is a continuation of application No. 12/157,823, filed on Jun. 13, 2008, now abandoned.

(51) Int. Cl. A61M 31/00

(52) U.S. Cl. CPC A61M 31/00
USPC 604/285

(57) ABSTRACT

A vaginal therapeutic device having an exterior surface of copper metal that has a configuration for insertion in and retention in the vagina, and utilizes the copper metal as an antimicrobial agent to therapeutically treat abnormal biological conditions in the vagina. The exterior configuration of the device defines a plurality of protuberances to resist expulsion of the device from the vagina.

### CROSS-REFERENCE TO RELATED PATENT APPLICATIONS

The subject patent application is a continuation-in-part of U.S. patent application Ser. No. 13/317,230 filed Oct. 12, 2011 which is a continuation of U.S. patent application Ser. No. 12/157,823 filed Jun. 13, 2008. The entire disclosures of U.S. patent applications Ser. No. 12/157,823 and Ser. No. 13/317,230 are incorporated herein by reference.

### BACKGROUND OF THE INVENTION

1. Field of the Invention

The present invention relates to therapeutic treatment of abnormal conditions in the vagina caused by microbial organisms and, more particularly, to such treatment utilizing a vaginal therapeutic device having an exterior surface made of copper metal and a shape to enhance retention of the device in the vagina.

2. Brief Discussion of the Related Art

In the area of female personal, hygiene and gynecological health, many efforts have been made to reduce microbial organisms in the vagina which create abnormal conditions such as vaginal odor, bacterial and viral infections, yeast infections and the like. Most of the attempts have involved medication and/or douching. Particularly effective douching systems utilizing a stainless steel douche appliance and methods using metal oxides are described in. U.S. Pat. No. 6,190,365, No. 6,589,216, No. 7,276,056 and No. 7,270,653 to Abbott et al. Abnormal vaginal conditions are caused by various microbial organisms including bacterial, viral and fungal organisms. No device or associated method has been found to provide an anti-microbial treatment to kill such microbial organisms. The current standard treatment for bacterial vaginosis (BV) is a drug with a reported cure rate of 90+ percent in 1993. Currently, the cure rate is far less and is falling, while the reoccurrence rate is increasing. The side effects of such drugs are numerous, and there is a need for a solution to this worsening problem.

It is known that many bacteria identified as human pathogens cannot survive on surfaces of copper metal. As used herein, “copper metal” means pure copper and copper alloys such as brasses, bronzes; copper-nickels and copper-nickel-zincs as described in “Abstract from Copper Alloys for Human Infection Disease Control” by H. T. Michaels, S. A. Wilks, J. O. Noyce and C. W. Keivel. The number of live bacteria drops from several orders of magnitude to almost zero on copper metal in a few hours. Copper metals which have been tested include high (pure) coppers, brasses, bronzes, copper-nickels, and copper-nickel-zincs. The bacteria tested include Methicillin-resistant staphylococcus aureus (MRSA), the cause of serious hospital-acquired infections, pseudomonas aeruginosa enterbacter aerogens and Acinetobartor baumonaii, as well as E. coli 0157:H7 and listeria monocytogenes, foodborne pathogens associated with several large-scale food recalls. Copper metal as a “static” agent inhibits microbial growth by means other than killing such that it limits the growth of microorganisms and may inactivate them. Copper metal as an “antimicrobial” substance (chemical or physical) can prevent microbial growth either by some “-static” action or by the outright killing of microbial organisms. Copper metal as a “-cidal” agent either damages a microbial organism at low concentration and/or reduced contact time or interacts permanently so that it ceases to function normally. Such “-cidal” agent damages a microorganism sub-lethally. Total inactivation is functionally equivalent to killing the organism (0% survival).

### SUMMARY OF THE INVENTION

In accordance with the present invention, a therapeutic device having a shape to be received and retained in the vagina has a copper metal exterior surface such that microbial organisms in the vagina are killed by the copper metal. In this manner, various bacterial conditions, fungal conditions and viral conditions are treated.

In one aspect, the present invention relates to a therapeutic device formed of a body having an exterior surface made of copper metal, the body having a shape including spaced bulbous projections or protuberances to resist expulsion of the device from the vagina.

In another aspect, the present invention treats abnormal biological conditions in the vagina by inserting a therapeutic device having an exterior surface made of anti-microbial copper metal in the vagina, retaining the therapeutic device in the vagina for a time sufficient to kill microbial organisms and thereafter removing the therapeutic device from the vagina.

Other aspects and advantages of the present invention will become apparent from the following description of the preferred embodiments taken in conjunction with the accompanying drawings.

### BRIEF DESCRIPTION OF THE DRAWINGS

FIG. 1 is a perspective view of a vaginal therapeutic device according to the present invention.

FIG. 2 is a side view, partly in section, of the vaginal therapeutic device of FIG. 1.

FIG. 3 is a side view of an alternative vaginal therapeutic device according to the present invention.

FIG. 4 is an exploded view, partly in section, of the alternative vaginal therapeutic device of FIG. 3.

FIG. 5 is a side view of the vaginal therapeutic device of FIG. 1 positioned in the vagina.

FIG. 6 is an enlarged side view depicting the manner in which the shape of the vaginal therapeutic device resists expulsion from the vagina and enhances retention of the device in the vagina.

### DETAILED DESCRIPTION OF THE INVENTION

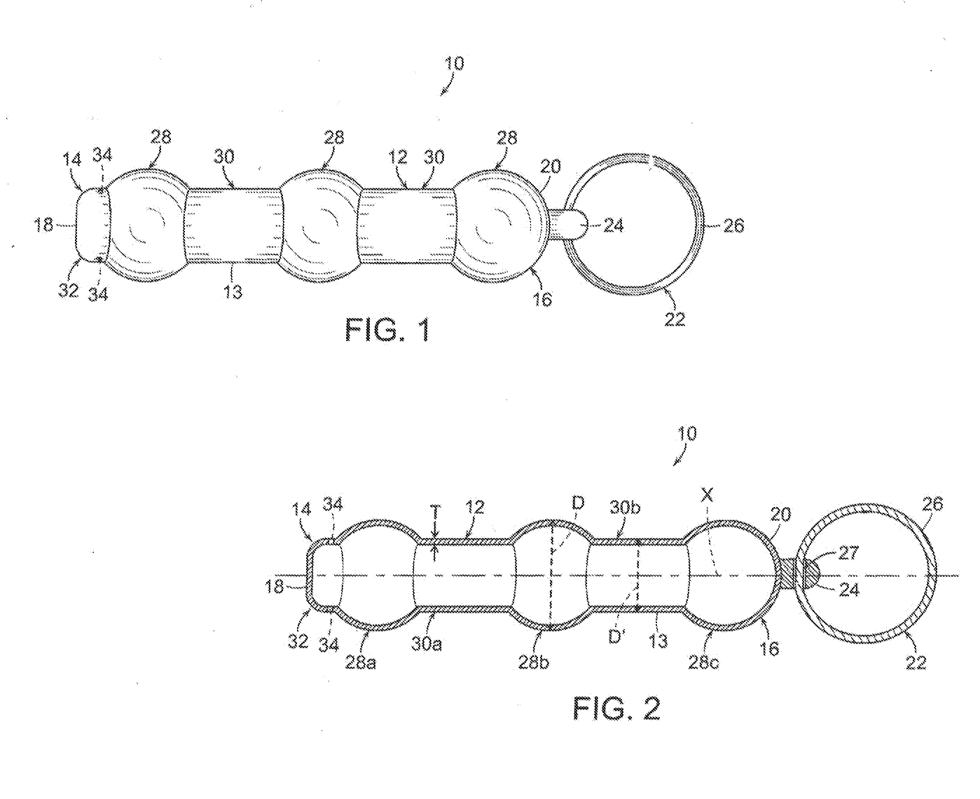

A vaginal therapeutic device 10, as shown in FIGS. 1 and 2, is formed of a rigid body 12 having a rounded exterior shape, circular or cylindrical in cross-section, with a continuous exterior surface 13 made of copper metal. As noted above, “copper metal” as used herein means pure copper (99.5% or greater copper) and copper alloys such as brasses, bronzes, copper-nickels and copper-nickel-zinc. The body 12 can be solid, but it is preferred that the body 12 be non-solid or hollow for reduced weight as shown in FIG. 2. The body 12 has a distal end 14, a proximal end 16 and a central longitudinal axis X.

The distal end 14 has a rounded, smooth shape to facilitate insertion of the body 12, distal end 14 first, in the vagina and to avoid trauma to anatomical tissue during insertion of the body 12 in the vagina and while the body 12 remains positioned in the vagina. In the case of body 12, the distal end 14 has a blunt cylindrical shape with a rounded convex distal end surface or wall 18. The proximal end 16 for body 12 is rounded and has a partial spherical or convex proximal end surface or wall 20. The vaginal therapeutic device 10 further includes a retraction structure or handle 22 carried at the proximal end 16 of body 12 to facilitate insertion of the body 12 in the vagina and removal of the body 12 from the vagina. The retraction structure 22 includes a stem 24 extending longitudinally in the proximal direction from the proximal end surface 20 and a ring 26 pivotally mounted to the stem 24. The stem 24 has a cylindrical configuration extending longitudinally from the proximal end surface 20 coaxial with the central longitudinal axis X and terminating at a rounded convex end. As seen in FIG. 2, a bore 27 is formed through the stem 24 transverse or perpendicular to the central longitudinal axis X, and the ring 26 extends through the bore 27 in order to pivotally mount or attach the ring 26 to the stem 24. In the case of vaginal therapeutic device 10, the stem 24 and ring 26 are made of copper metal. However, it should be appreciated that the stem 24 and/or the ring 26 may be made of a material different from the material of the body 12, such as being made of a lighter weight non-metal material which can also be flexible, for example plastic. It should also be appreciated that the retraction structure or handle 22 can be designed in various alternative ways and with various alternative structures made of the same material as body 12 or different materials than the body 12.

The exterior shape or configuration of body 12 includes a plurality of longitudinally spaced bulbous projections or protuberances 28 between distal end 14 and proximal end 16, and a connecting portion 30 disposed between each pair of adjacent, longitudinally spaced protuberances 28. A protuberance 28 may be disposed at distal end 14 and/or proximal end 15, such that the distal end 14 and/or the proximal end 16 may be formed by a portion of the protuberance. Body 12 is formed with three protuberances 28, i.e. a distal protuberance 28a, a middle protuberance 28b and a proximal protuberance 28c, the middle protuberance 28b being disposed between the distal and proximal protuberances 28a and 28c. The protuberances 28 are coaxial with the central longitudinal axis X of the body 12. Each protuberance 28 is substantially spherical in exterior configuration and has a major transverse exterior dimension D, transverse or perpendicular to the central longitudinal axis X, corresponding to its spherical outer diameter. In the case of body 12, the major transverse dimensions D of the protuberances 28 are the same. Although three protuberances 28 are preferred, it should be appreciated that the body 12 could have two protuberances or more than three protuberances.

Since the body 12 has three protuberances 28, the body 12 has two connecting portions 30, i.e. a distal connecting portion 30a extending longitudinally between distal protuberance 28a and middle protuberance 28b, and a proximal connecting portion 30b extending longitudinally between middle protuberance 28b and proximal protuberance 28c. It should be appreciated that, depending on the number of protuberances, the body 12 can have one or more connecting portions 30. Each connecting portion 30 has a cylindrical configuration with opposed ends joining adjacent longitudinally spaced protuberances 28. Each connecting portion 30 has its longitudinal axis coaxial with the central longitudinal axis X. Each connecting portion 30 has a transverse exterior dimension D′, transverse or perpendicular to the central longitudinal axis X, corresponding to its cylindrical outer diameter. In the case of body 12, the transverse dimensions D′ for the connecting portions 30 are the same. The transverse dimensions D′ for the connecting portions 30 are less than or smaller than the major transverse dimensions D for the protuberances 28, such that the protuberances 28 bulge outwardly in the transverse direction beyond the connecting portions 30. In other words, the major transverse dimensions D of the protuberances 28 are greater than the transverse dimensions D′ of the connecting portions 30, such that the connecting portions 30 define narrower portions of the body 12 between the protuberances 28.

In the case of body 12, the proximal end 16 and the proximal end surface or wall 20 are formed by a portion of the exterior surface of the proximal protuberance 28c. The distal end 14 for body 12 is formed by a cylindrical extension or nose 32, similar to a connecting portion 30 but of shorter length, extending longitudinally in the distal direction from the distal protuberance 28a and terminating at the distal end surface or wall 18. It should be appreciated, however, that the distal end 14 and the distal end surface or wall 18 can be formed by a portion of the exterior surface of the distal protuberance 28a as explained herein for vaginal therapeutic device 110. The nose 32 is coaxial with the central longitudinal axis X and has a transverse exterior dimension smaller than the major transverse dimension D, the nose 32 having a transverse exterior dimension D′.

Due to the weight of copper metal, it may be preferable for the body 12 to be hollow with a wall thickness T as shown in FIG. 2; however, the body 12 can be solid if desired. Additionally, it is preferred that the copper metal be pure copper, i.e. 99.95% copper after processing. However, while pure copper has the greatest anti-microbial or therapeutic effect, various copper alloys as described above can be utilized. The walls of the body 12 can be entirely copper metal. Alternatively, the body 12 can have an inner part made of a less expensive, lighter weight material, such as plastic, with the inner part carrying an outer layer formed of copper metal that defines the exterior surface of the body 12.

To fabricate the vaginal therapeutic device, it is noted that copper metal can be readily electro-formed, plated, hot rolled, extruded, cast or forged and can be cold rolled to a desired thickness to form sheets or layers. The vaginal therapeutic device 10 can, for example, be made of one piece or two or more welded pieces. The body 12 could, for example, be fabricated from two pieces or parts, each part being stamped from a sheet of copper metal to have a shape corresponding to one half of the body 12 when bisected by a plane containing the central longitudinal axis X, the parts being welded together along a longitudinal weld aligned with the central longitudinal axis X to complete the configuration of the body. Alternatively, the body 12 could be fabricated from individual parts corresponding. respectively, to the protuberances 28, the connecting portions 30, the stem 24, and the nose 32, with the individual parts being welded together via circumferential welds at the ends of the parts to complete the configuration of the body. When parts are welded together to form the body, it is preferred that the copper metal itself serve as the weld material without introducing an extraneous weld material. Depending on the process used to manufacture the vaginal therapeutic device, it may be necessary to provide vent holes through the wall of the body to relieve internal pressure. Accordingly, as seen in FIG. 2, vent holes 34 can be formed in the nose 32 adjacent the distal protuberance 28a, with the holes 34 being located at spaced radial locations about the central longitudinal axis X. Another suitable location for one or more vent holes would be distal end wall 18 and/or proximal end 16.

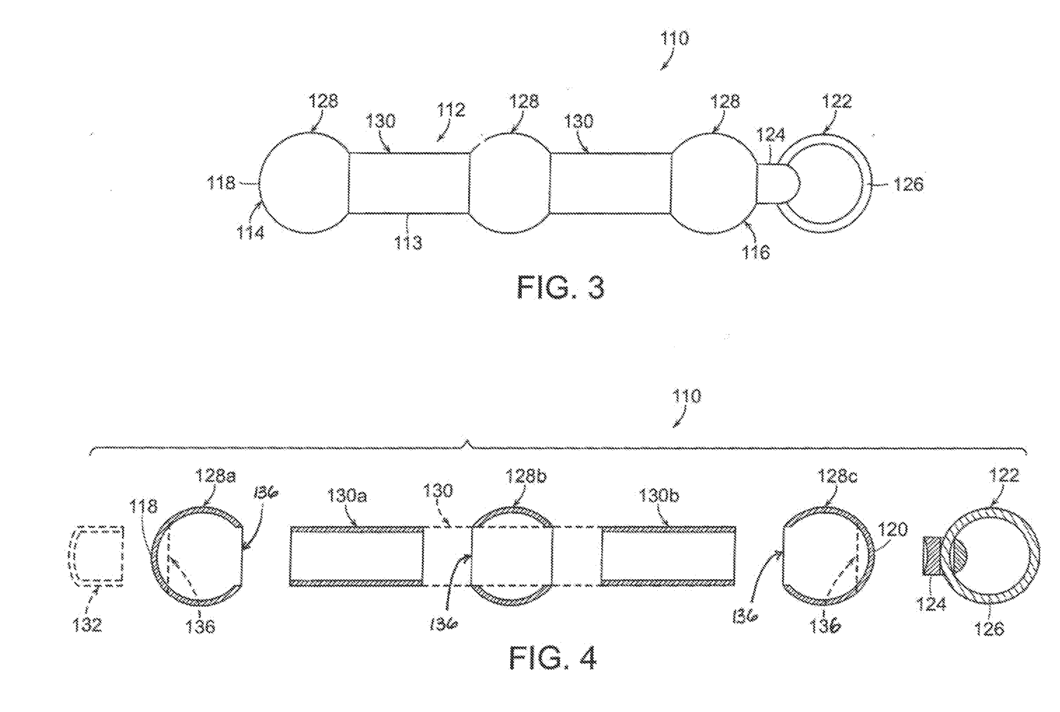

FIGS. 3 and 4 illustrate an alternative vaginal therapeutic device 110. The vaginal therapeutic device 110 is similar to the vaginal therapeutic device 10, but is an example of one in which the distal end 114 and distal end surface or wall 118 are formed by part of the exterior spherical surface of the distal protuberance 128a. In addition, the vaginal therapeutic device 110 is an example of one in which the body 112 is fabricated from individual parts corresponding, respectively, to the distal, middle and proximal protuberances 128a, 128b and 128c, the distal and proximal connecting portions 130a and 130b, and the retraction structure 122. The protuberances 128a, 128b and 128c are spherical in exterior configuration except for an appropriately located and sized transverse opening 136 at one or both ends to join or mate with the end of a connecting portion 130. In the case of body 112, the middle protuberance 128b has openings 136 along its distal and proximal sides to join or mate with ends of connecting portions 130a and 130b, respectively. The distal protuberance 128a has an opening 136 along its proximal side to join or mate with the opposite end of connecting portion 130a. The proximal protuberance 128c has an opening 136 along its distal side to join or mate with the opposite end of connecting portion 130b. Each protuberance 128 can be fabricated from two semi-spherical or partial spherical sections that are welded together to form the protuberance. The parts forming the protuberances 128 and the connecting portions 130 can be welded together via circumferential welds to complete the configuration of the body 112. The stem 124 of the retraction structure 122 can be welded to the proximal end surface 120 of the proximal protuberance 128c. As shown in dotted lines in FIG. 4, the distal protuberance 128a can optionally be provided with a transverse opening 136 along its distal side to join or mate with the nose 132 along a circumferential weld, in which case the nose 132 defines the distal end surface 118 as described for vaginal therapeutic device 10. Also, where it is unfeasible to attach the stern 124 to the proximal end surface 120, the proximal protuberance 128c may be provided with an opening 136 along its proximal side, as shown in dotted lines in FIG. 4, to join or mate with the stem 124. It should be further appreciated that the connecting portions 130a and 130b can be formed as a single connecting portion 130, as shown in dotted lines in FIG. 4, that can pass through the openings 136 in middle protuberance 128b such that opposite ends of the single connecting portion join or mate with the openings in the distal and proximal protuberances 128a and 128c, respectively.

It is preferred that the body 12, 112, excluding the retraction structure 22, 122, have a length in the range of 3.125 to 3.25 inches; that the major transverse dimension D of the protuberances 28, 128 be in the range of 0.625 to 0.75 inch; that the transverse dimension D′ of the connecting portions 30, 130 be in the range of 0.375 to 0.500 inch; that the length of the connecting portions 30, 130 be 0.831 inch or substantially 0.831 inch; that the wall thickness T of the body 12, 112 be in the range of 0.031 to 0.032 inch; that the length of the stem 24, 124 be in the range of 0.271 to 0.276 inch; that the diameter of the stem 24, 124 be 0.25 inch or substantially 0.25 inch; that the outer diameter of the ring 26, 126 be in the range of 0.625 to 0.875 inch; that the inner diameter of the ring 26, 126 be in the range of 0.5 to 0.715 inch; and that the length of nose 32, 132 be 0.279 inch or substantially 0.279 inch.

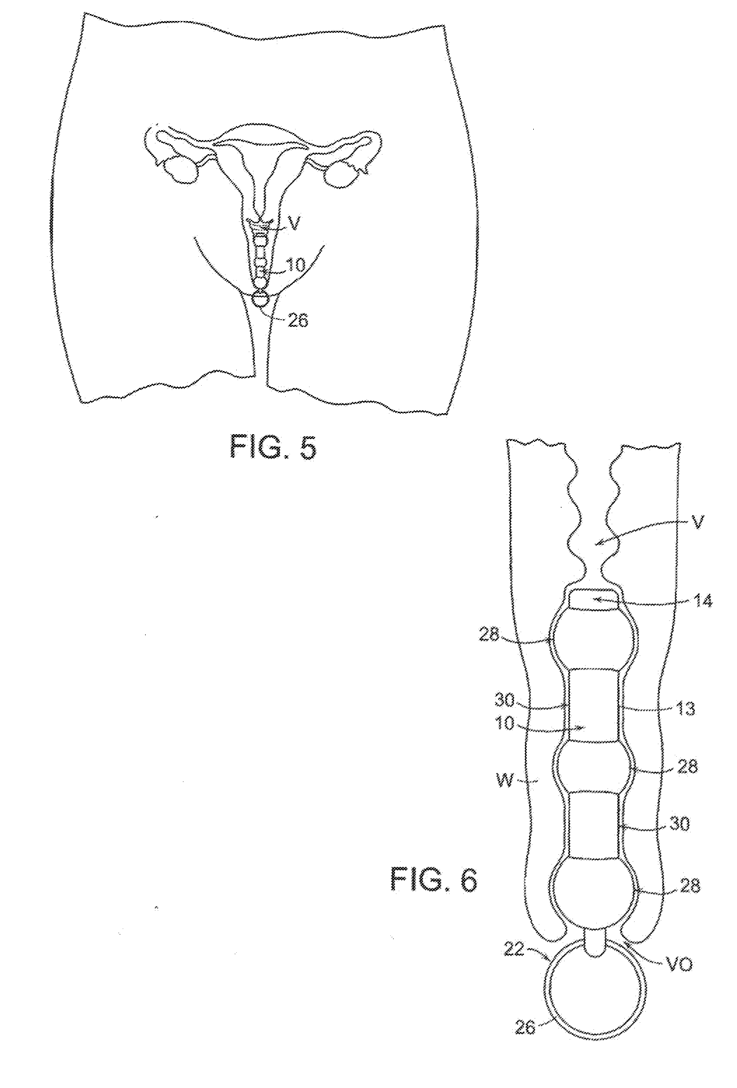

FIGS. 5 and 6 illustrate the manner in which the vaginal therapeutic devices are used in a method of treating the vagina for abnormal biological conditions. Although FIGS. 5 and 6 depict the vaginal therapeutic device 10, it is noted that the vaginal therapeutic device 110 is used in the same manner. In use, the vaginal therapeutic device 10 is inserted, distal end 14 first, in the vagina V, i.e. the vaginal canal, through the vaginal opening VO as shown in FIGS. 5 and 6. The elastic nature of the wall W of the vagina V allows the vagina to expand in order to receive the device 10 therein. The rounded exterior configuration of the body 12 and its distal end 14 facilitates insertion of the device 10 in the vagina V and avoids trauma to anatomical tissue during insertion of the device in the vagina V and while the device remains inserted within the vagina V. During insertion, the user will typically grasp the ring 26 of retraction structure 22 by the hand in order to gently push the vaginal therapeutic device 10 into the vagina V. The narrow transverse dimension of the distal nose 32 further facilitates insertion and advancement of the device in the vagina. The vaginal therapeutic device 10 is advanced into the vagina V by the user until the ring 26 is located near the vaginal opening VO while still protruding from the vaginal opening. The elastic nature of the wall W of the vagina causes the wall W to contract and deform along the device 10 so as to contact the exterior surface 13 of the vaginal therapeutic device as best seen in FIG. 6. As a result of the vaginal wall W contracting into contact with the exterior surface 13 of the body 12, the protuberances 28 function to resist expulsion of the vaginal therapeutic device 10 from the vagina V and enhance retention of the vaginal therapeutic device 10 within the vagina V. Contact of the wall W of the vagina with the exterior surface 13 of the vaginal therapeutic device causes microbial organisms to contact the copper metal such that the microbial organisms are killed. The time required to, kill the microbial organisms depends on the purity of the copper metal; however, if the device 10 is inserted in the vagina at bedtime and allowed to remain inserted in the vagina overnight, i.e. for 6 to 8 hours or the time equivalent to a normal night's sleep, essentially all microbial organisms will be killed by the time the vaginal therapeutic device is removed from the vagina in the morning. To remove the vaginal therapeutic device 10, the user need only grasp the ring 26 of the retraction structure 22 by the hand and gently pull with sufficient force to overcome the retention force of the protuberances 28 in contact with the wall W. The rounded shape of the body 12 and the protuberances 28 facilitates removal of the vaginal therapeutic device 10 and avoids trauma to anatomical tissue during removal of the vaginal therapeutic device. In order to treat abnormal biological conditions in the vagina, it is preferred that the vaginal therapeutic device 10 be used daily for 6 to 8 hours, or the time equivalent to a normal night's sleep, for seven consecutive days. Therefore, a preferred method of use involves inserting the vaginal therapeutic device in the vagina at bedtime, allowing the device to remain positioned in the vagina overnight, or preferably six to eight hours, removing the device from the vagina in the morning, and repeating the procedure for seven consecutive nights.

When a vaginal therapeutic device according to the present invention is inserted or positioned in the vagina, the antimicrobial effect of the copper metal will kill or inactivate and inhibit growth of microbial organisms in the vagina which cause abnormal conditions, including viruses, bacterium, fungi and the like. Thusly, undesirable conditions such as odor, vaginitis, yeast infections, bacterial infections, viral infections, sexually transmitted diseases and the like are treated, cured or prevented, it being noted that HIV (human immunodeficiency virus), which causes AIDS, can be treated in accordance with the subject invention in that a vaginal echo-system imbalance caused by microbial organisms causes the vagina to be receptive to HIV. In addition, the vaginal therapeutic device can be used to provide a contraceptive or spermicidal effect, in that sperm may be killed or rendered inactive by the copper metal and/or the copper metal may act to create a vaginal environment that is inhospitable to sperm.

In as much as the present invention is subject to many variations, modifications and changes in detail, it is intended that all subject matter discussed above or shown in the accompanying drawings be interpreted as illustrative only and not to be taken in a limiting sense.

What is claimed is:

1. A vaginal therapeutic device comprising
a rigid body having an exterior surface made of copper metal, said body having a rounded distal end, a proximal end, and a central longitudinal axis, said body including a plurality of longitudinally spaced protuberances between said distal end and said proximal end, and at least one connecting portion extending between adjacent protuberances, said protuberances having a transverse dimension perpendicular to said central longitudinal axis, and said at least one connecting portion having a transverse dimension perpendicular to said central longitudinal axis, said transverse dimension of said protuberances being greater than said transverse dimension of said at least one connecting portion, said body being insertable, distal end first, in the vagina such that the wall of the vagina contacts said exterior surface and said protuberances resist expulsion of said body from the vagina, whereby said copper metal in contact with the wall of the vagina acts as an antimicrobial agent to therapeutically treat abnormal biological conditions arising in and/or affecting the vagina.

2. The vaginal therapeutic device as recited in claim 1 wherein said device further comprises retraction structure carried at said proximal end of said body.

3. The vaginal therapeutic device as recited in claim 1 wherein said exterior surface is rounded, with said protuberances and said at least one connecting portion having a circular configuration in cross-section.

4. The vaginal therapeutic device recited in claim 1 wherein said body is hollow.

5. The vaginal therapeutic device recited in claim 1 wherein said body includes a distal protuberance, a proximal protuberance and a middle protuberance disposed between said distal protuberance and said proximal protuberance, said body includes a distal connecting portion extending longitudinally between said distal protuberance and said middle protuberance, and a proximal connecting portion extending longitudinally between said middle protuberance and said proximal protuberance.

6. The vaginal therapeutic device recited in claim 5 wherein each of said protuberances has a substantially spherical configuration coaxial with said central longitudinal axis, said transverse dimension of said protuberances corresponds to the spherical diameter of said protuberances, each of said connecting portions has a cylindrical configuration coaxial with said central longitudinal axis, and said transverse dimension of said connecting portions corresponds to the cylindrical diameter of said connecting portions.

7. The vaginal therapeutic device recited in claim 6 wherein said proximal end is formed by a convex surface of said proximal protuberance.

8. The vaginal therapeutic device recited in claim 7 wherein said distal end is formed by a cylindrical nose extending distally from said distal protuberance to terminate at a rounded distal end surface, said cylindrical nose having a transverse dimension transverse to said central longitudinal axis that is less than said transverse dimension of said protuberances.

9. The vaginal therapeutic device recited in claim 7 wherein said distal end is formed by a convex surface of said distal protuberance.

10. The vaginal therapeutic device recited in claim 7 wherein said vaginal therapeutic device further comprises a retraction structure including a ring pivotally attached to said proximal protuberance.

11. The vaginal therapeutic device recited in claim 1 wherein said copper metal is pure copper.

12. The vaginal therapeutic device recited in claim 1 wherein said copper metal is a copper alloy.
